(19) United States
(12) Patent Application Publication
(10) Pub. No.: US 2025/0082492 A1
(43) Pub. Date: Mar. 13, 2025

(54) POST-NATAL GLUTEAL ORTHOSIS

(71) Applicant: Eurl Cornier, Paris (FR)

(72) Inventor: Edgard CORNIER, Paris (FR)

(21) Appl. No.: 18/727,463

(22) PCT Filed: Dec. 12, 2022
(86) PCT No.: PCT/FR2022/052316

§ 371 Date: Jul. 9, 2024

(30) Foreign Application Priority Data
Jan. 10, 2022 (FR) FR2200148
Nov. 23, 2022 (FR) FR2212223

(51) Int. Cl. A61F 5/28

(52) U.S. Cl. CPC A61F 5/28

(57) ABSTRACT

Post-natal gluteal orthosis (100) comprising a belt (1) carrying two holding devices (2) fixed to the rear part (1A) of the belt (1) and each consisting of: a triangular piece (21) fixed by its base to the belt (1), extending into a thinned part (22) connected to a strap (23) terminating in an adjustable hooking device (24) for adjustable fixing to a fixing point (12) at the front (IAV) of the belt {1). The tapered part (22) and the strap (23) pass over the perineum to cross-join the respective attachment point (12) to the front part (IAV) of the belt.

### FIELD OF INVENTION

The present invention relates to a post-natal gluteal orthosis.

### STATE OF THE ART

The postulate that governs all reasoning relating to the physiology of the perineum is that of vertical pressure from the abdomen and the earth's gravity. These combined pressures are supported by the perineal floor. This postulate is considered as without any justification. But no post-natal orthosis could be described on the basis of this postulate. The inventor developed a new postulate according to which the perineum supports horizontal pressures and displacements. This postulate is developed under the term «vectorial perineology»: http.//www.perineologie-vectorielle.com.

Known devices are vertical. However, any vertical pressure perpendicular to its direction has the effect of distending the perineal floor. Nearly 40% of women who give birth have scarring in the wrong position.

### PURPOSE OF THE INVENTION

The aim of the present invention is to develop a device for supporting the gluteal muscles which pushes forward the perineum dis-tensioned, making it possible to develop horizontal pressure on the perineum to ensure its closure by allowing the osteo ligamentary, muscular and fibrous ensemblement to be held in place during the healing period. Another aim of the invention is to straighten out all the internal osseous, ligamentary and muscular spacing that accompanies the healing process.

The perineum and the whole of the small pelvis must adapt to the moment of birth.

### DESCRIPTION AND ADVANTAGES OF THE INVENTION

To this end, the invention relates to a post-natal gluteal orthosis comprising:
a belt to support the sacroiliac and pubic joints, as well as a sub-abdominal sheathing effect.
two holding devices
   fixed to the back of the belt
   each consisting of:
a triangular piece attached at the base to the belt,
extending into an amine part and a lanyard connected to a hooking device for adjustable attachment to a fixing point
at the front of the belt
the two holding devices crossing behind the perineum to create a horizontal forward thrust,
the tapered part and the strap passes behind the perineum to cross over the respective attachment point at the front of the belt.

The brace used in this invention maintains the patient in the healing position by compressing the perineal tissues. The pressure indirectly constricts and compresses the perineum. This facilitates the healing of ruptured tissues and fractured edges.

According to the invention, the belt consists of a rear part in the form of a loop in one plane, connected on each side by a respective intermediate part, these intermediate parts being curved downwards and symmetrical to join the front part at the level of the pubis below the waist the abdomen; these parts curve downwards and then upwards to form a basket handle when joined together.

The belt and the sub-abdominal sheathing help to push upwards the Core of the small pelvis (Core according to J PILATE).

Another feature of the orthosis is that it includes a pad that is associated with each support device and is removably and adjustably attached to it in the area around the intersection of the two support devices.

The two adjustable pads are positioned in the space between the two ischial tuberosities and the tip of the coccyx. Each support device exerts a horizontal forward thrust on the tissues via the two pads.

According to the invention, each of the two holding devices is directed medial to the trochanteric masses, crossing behind the pelvic opening and then forward.

Another feature of the triangular piece is that it is made of an elastic fabric.

Another feature is the elasticity of the triangular part.

Another feature the axis is oriented so that elasticity is mainly in the transverse direction and low in the longitudinal direction.

In another feature, the adjustable hooking device is a buckle through which the end of the strap passes, this buckle being fixed or secured to the fixing point in the front part of the belt.

### BRIEF DESCRIPTION OF THE DRAWINGS

The present invention will be described in greater detail below with the aid of an example of an orthosis shown in the figures in which:

FIG. 1 developed flat view of the orthosis according to the invention

FIG. 2A rear view of brace in place

FIG. 2B front view of brace in place

FIG. 3 rear view of the orthosis installed on a mannequin

FIG. 4 perspective view of another embodiment of an orthosis according to the invention,

FIG. 5 view of the second method of fitting the belt on the pelvis,

FIG. 6 perspective view of the belt alone.

### DESCRIPTION OF AN EMBODIMENT

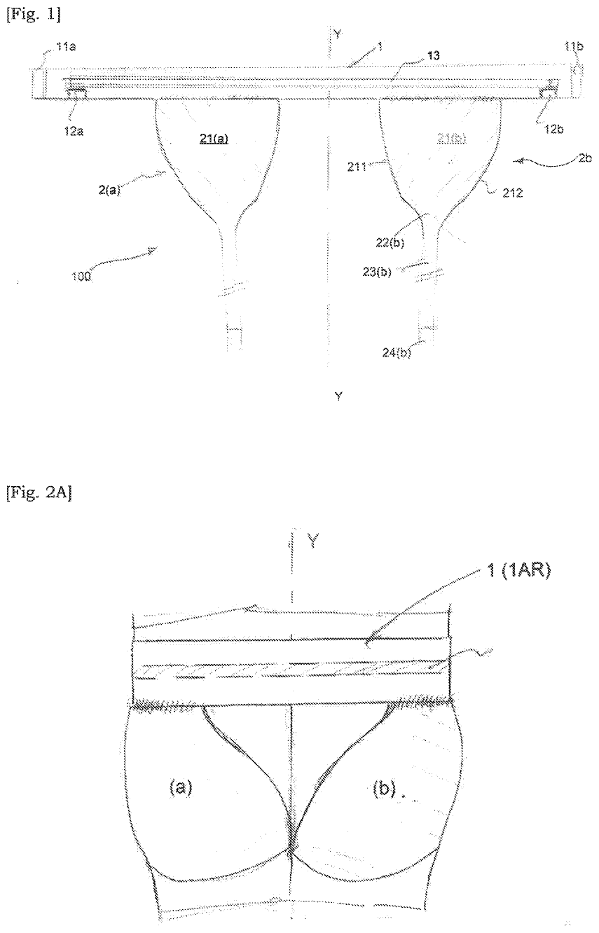

According to FIG. 1, the object of the invention is a post-natal gluteal orthosis tale 100 presented in its deployed state.

The brace 100 consists of a belt 1 used for support by the patient which the orthosis is attached to the subject. The belt 1 carries, in symmetrical positions relative to a median line YY, two holding devices 2 oriented in the longitudinal direction, which is substantially the median direction YY. This direction intersects the transverse direction represented by the belt 1 and is in particular perpendicular.

As the two holding devices 2 are symmetrical, the description will be limited to one of them. In order to simplify the description and not to clutter it up unnecessarily with complex references, the holding devices 2 have numerical references supplemented by a suffix a or b, 4 which will only be used to explain the crossing of the attachment of the two holding devices 2a, 2b.

The retaining device 2 consists of a triangular part 21 fixed to the belt 1 at its base and tapering in the opposite direction by a tapered part 22 connected to a strap 23 terminated by an adjustable hooking device 24.

The holding device 2 can be made in a single piece from of a fabric which is elastic in the transverse direction and which is not necessarily perpendicular to the direction of the belt. The tapered part 22 can retain a certain elasticity, which is substantially unidirectional, whereas the strap 23 is preferably ineradicable or very slightly elastic. The strap 23 ends in an adjustable attachment element 24, such as a micro-gripping segment, enabling the overall length of each holding device 2 to be adjusted.

The belt 1 carrying the two retaining devices 2 is an adjustable belt with two assembly ends 11, not shown in detail, corresponding to the front parts 2A, 2B of the belt.

The belt 1 is preferably formed by a reinforcement 13 such as a band of polycarbonate integrated into the body of the belt. As the band is not integral with the end buckles 11, the belt retains a certain elasticity of its own.

Preferably, the belt 1 of the device is reinforced along the entire length of the belt along the back three quarters of its circumference by the band of soft, flexible material under the iliac crests, but strong vertically, for example a thin sheet of polycarbonate. This prevents the belt from bending.

Adjustment of the length of the belt 1 is illustrated by the two connecting elements at the ends 11 of the belt.

Close to the ends, the belt 1 has two attachment points 12 for receiving see the ends 24 of the strips 23 after crosswise positioning of the straps of the brace 100.

The holding devices 2a, b are crossed diagonally to direct the forces exerted by the triangular parts 21a, 21b.

The triangular part 21 itself has a non-symmetrical shape, the inner side 211 being relatively straight or slightly curved and the outer side 212 being highly curved to create the contour of the enclosing shape.

So each triangular piece 21a, 21b has a wide bow attached to the side of the waistband. It runs towards the trochanteric mas-sifs, to come to rest in front of the ischial tuberosities. At the level of the perineum, the fabric narrows to form a strap 23.

This strap crosses the strap coming from the opposite side. As shown, the strap joins the front circumference of the belt 1. The length of the strap can be adjusted so that its tension can be adjusted.

The fabric of the triangular pieces 21a, 21b has a double elasticity. It is wide perpendicular to the direction of the straps 23 so as to encompass each buttock mass. The elasticity is low along the length of the triangular part, so that its tension can be adjusted.

The distance of the holding devices 2a, 2b from the median line YY in the transverse direction depends on the anatomical characteristics of the subject.

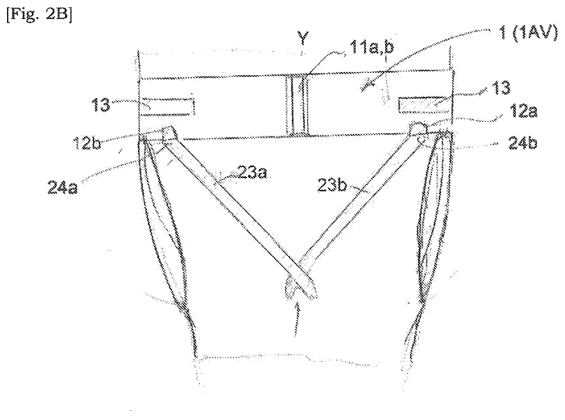

FIGS. 2A and 2B show the fitting of the orthosis and the crossing of the orthosis the two holding devices 2a, 2b.

At the front (FIG. 2B) the crossed straps 23a, b join the fastening points 12b, a on the belt.

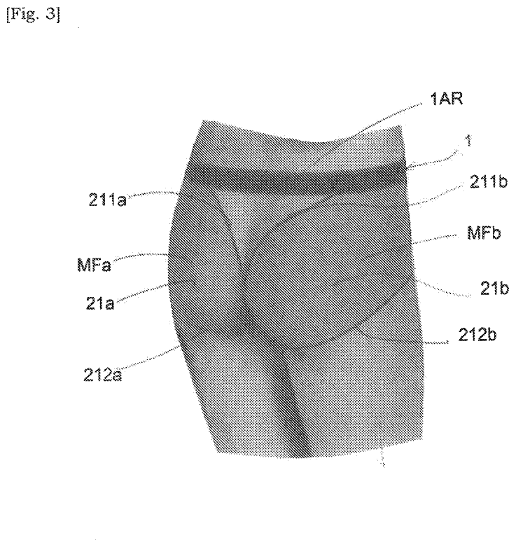

FIG. 3 is a perspective view of a dummy on which the brace 100 has been fitted.

FIG. 3 shows the shape of the two triangular pieces 2a, b and the attachment of the two triangular pieces 21a, b to the front part 1AR of the belt. The contours are labelled 211a, b; 212a, b.

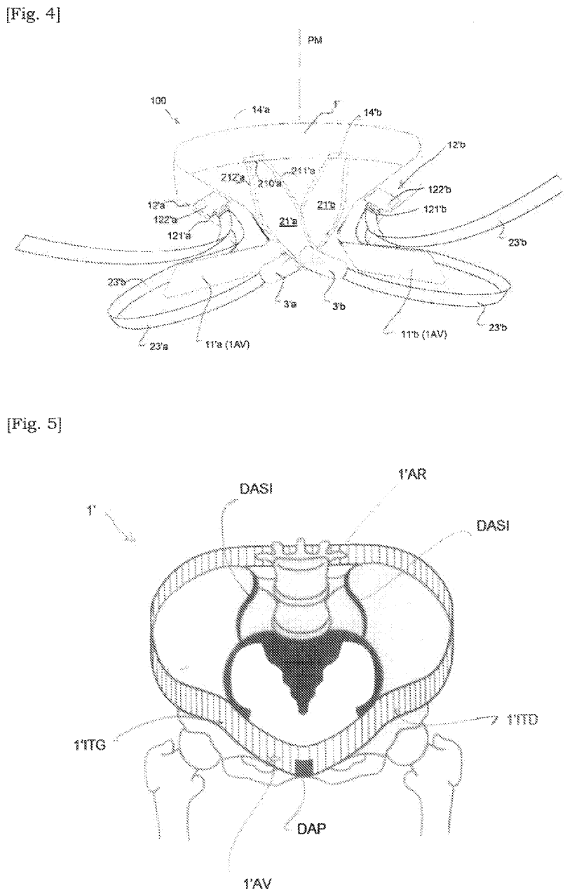

According to FIG. 4, the other embodiment 100′ of the post-natal orthosis will be described below. Elements with the same function as the elements of the embodiment described above will bear the same alphanumeric references supplemented by ( ) which will only be associated with the elements of the embodiment described above numerical references of the claims only if this is necessary for understanding.

In order not to complicate the presentation of this embodiment, the references used in the description and the claims will not be repeated not completed by the suffixes (a) and (b) that these numerical references bear in FIG. 4.

FIG. 4 shows the post-natal gluteal orthosis 100′ in the following view in perspective in the unfolded state, with a symmetrical structure with respect to the median plane perpendicular to the plane of the figure and schematised by its trace PM.

The brace 100′ consists of a belt 1 which, in positions symmetrical with respect to the median plane PM, two holding devices 2′ oriented in a crossed position. The two holding devices 2′ are symmetrical, so the description will be limited to one of them, using the same alphanumeric reference conventions as in the first embodiment.

Each holding device 2′ consists of a triangular or triangular-like part 21 with curved sides. Part 21′ is fixed by its base 210′ to the belt 1′. This part 21 tapers overall to a tapered part 22′, which is continued by a narrower part 22′, which is continued by a narrower part 22′, which is continued by a narrower part 22′ 23′. The triangular part has elasticity in the transverse direction which, as in the first embodiment, is not necessarily the direction perpendicular to the direction of the belt or perpendicular to the median plane PM.

The tapered part 22′ can retain a certain elasticity in one direction, whereas the strap 23′ which continues the tapered part 22′ is preferably inextensible or only slightly elastic.

The strap 23′ passes through an attachment element of the fixing point front 12′. This attachment point allows the buckle 121 to pivot to adapt to the orientation imposed on the layout of the holding device 2′ and in particular the strap 23′ by the morphology of the subject.

The attachment point 12′ preferably comprises a loop 121 in through which the strap 23′ is passed and locked in an adjustable manner. This buckle 121 is, for example, a strap buckle, cam buckle, slide buckle or self-locking buckle, connected to the attachment point 12′ of the belt 1′.

The end of the strap 23′ is advantageously provided with a hooking surface such as a micro-scratch surface to hold the end to the belt.

As in the first embodiment, the belt 1 can be reinforced for strength, with a reinforcement that does not extend to the ends where the belt closes, so as to retain a certain elasticity.

At the back and over two-thirds of its circumference, the 1′ belt is reinforced by a vertically rigid but horizontally flexible material, for example a thin sheet of polycarbonate, inserted into the fabric folded around it.

At the front 1′AV, the ends 11 are preferably fitted with elements fastening or hooking devices, such as micro-grips, enabling the belt to be adjusted or adapted to the waistline, while retaining the belt function, i.e. supporting the tensioned holding devices 2′.

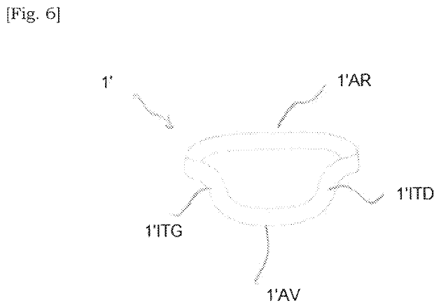

The particular three-dimensional shape of the belt 1 is shown in FIGS. 5 and 6.

According to the perspective view and the explanations previously given, the attachment points 12′ are located at the front and in positions in some way diametrically opposed to the rear fixings 14′ of the parts 21′ of each holding device 2′ so as to have a diagonal layout of the two holding devices 2′.

Beyond part 21′, at the tapered part 22′, each dis the positive holding device 2′ receives a pad 3′ detachably connected to the holding device 2′ to be located in the region surrounding the intersection of the two holding devices 2′ when they are worn by the subject.

These elongated pads 3′ preferably have detachable fastening elements on the underside, such as adhesive or micro-gripping surfaces, so that they can be attached in a removable and adjustable manner to each holding device 2′.

The positioning of the pads 3′ and the adjustment of the tension of the support dis-positives 2′ at the level of the straps 23′ passing through the plates allow the orthosis to be adapted to all morphologies for exerting the thrust as previously explained.

The loop-shaped fastening devices 121 form part of the fastening points 12′. However, it is also possible, as is generally the case, for the end of the strap 23′ to befitted with an adjustable hooking device which connects or hooks to the attachment point 12′ carried by the front part of the respective AV belt.

FIG. 5 shows the positioning of the waistband 1′ consisting of a rear part 1′AR continuing on each side with an intermediate part 1ITG, 17TD, curved downwards in a symmetrical Z and S shape to end with the front part AV at pubic level; the belt 1′ thus has a three-dimensional shape with the front 1′AV below the horizontal plane of the rear 1′AR.

The rear 1′AR part of the belt is worn over the under of the iliac crests of the bony pelvis.

The belt 1′ thus creates efforts to bring together and maintain the amplified bone joints, reducing sacroiliac joint distension and a sheathing that lifts the heart from the pelvic organs (Pilate core) by a hypopressive sheathing with a reduction in the pressure on the pubic joint distortions and support for the abdomen through the sheathing effect.

FIG. 6 shows the three-dimensional shape of the belt 1′ with its rear part I′AR looped in one plane, connected on one side by one Z-shaped intermediate part 1′TG and on the other side by the S-shaped intermediate part 1′ITD, symmetrical with respect to the vertical median plane not shown, to join the front part AV at pubic level. The assembly of the two front parts 11′A, 11′B of the belt 1 is not detailed in these FIGS. 5, 6.

The belt 1 is stiffened in the vertical direction to retain its three-dimensional shape when buckled and tensioned.

The criss-cross arrangement of the two holding devices 2 and their use in tension helps to maintain the three-dimensional shape of the 1′ frame.

### NOMENCLATURE OF MAIN COMPONENTS

100 Post-natal buttock orthosis
1 Belt
1AR Rear part of the belt
1AB Front part of the belt
11 Belt end
111, Buckle
12 fixing point
121 Loop
122 Leg
13 Reinforcement
14 Rear mounting
2, 2a, 2b Holding device
21 Triangular piece forming a bonnet
210 Base
211 Inside
212 Exterior side
22 Thinner part
23 Strap
24 Hanging device
DASI Distension of the sacroiliac joints
DAP Distortion of the pubic joints
1′ITG Intermediate part of the belt
1′ITD Intermediate belt section

1-9. (canceled)

10. Belt providing joint support for the sacroiliac and pubic joints, as well as a sub-abdominal sheathing effect,
characterised in that it consists of a rear part (1′ AR) in the form of a loop in one plane, connected on each side by a respective intermediate part (1′ ITG, 1′ ITD), these intermediate parts being curved downwards and symmetrical to join the front part (1′AV) at the level of the pubis below the subject's abdomen.

11. Belt according to claim 10,
characterised in that it is stiffened in the vertical direction to retain its three-dimensional shape when looped and tensioned.

12. Belt according to claim 10,
characterised in that it is provided with a reinforcement (13) in the form of a strip which is flexible longitudinally but rigid transversely.

13. Belt according to claim 12,
characterised in that the reinforcement (13) extends over the rear three quarters of the circumference of the belt.

14. Belt according to claim 10,
characterised in that the intermediate parts (the ITG and ITD) are curved downwards and then upwards in a basket-handle shape when they are joined together.

15. Post-natal gluteal orthosis (100),
characterised in that it comprises
a belt (1) providing joint support for the sacroiliac and pubic joints, as well as a sub-abdominal sheathing effect, characterised in that it comprises
two holding devices (2)
attached to the rear part (1AR) of the belt (1)
each consisting of:
a triangular part (21) attached by its base to the belt (1), extending into a tapered part (22) and a strap (23) connected to a hooking device for adjustable attachment to an attachment point (12) on the front (1AV) of the belt (1)
the two holding devices crossing behind the perineum to create a horizontal forward thrust,
the tapered part (22) and the strap (23) pass behind the perineum to join crosswise the respective attachment point (12) at the front part (1AV) of the belt.

16. Orthosis according to claim 15,
characterised in that the belt is a belt according to claim 10.

17. Orthosis (100) according to claim 16,
characterised in that it comprises a pad (3) associated with each holding device (2) and removably and adjustably fixed to each holding device so as to be in the space between the two ischial bone tuberosities of the subject.

18. Orthosis (100) according to claim 16,
characterised in that the triangular part (21) is made of an elastic fabric.

19. Orthosis (100) according to claim 18,
characterised in that the elastic fabric of the triangular part (21) is oriented so that the elasticity is mainly in the transverse direction and low in the longitudinal direction (YY).

20. Orthosis (100) according to claim 16,
characterised in that the adjustable fastening device is a buckle (121) through which the end of the strap (23) passes, this buckle being connected or fixed to the fastening point (12) of the belt.
